(19) United States
(12) Patent Application Publication
(10) Pub. No.: US 2023/0329604 A1
(43) Pub. Date: Oct. 19, 2023

(54) Blood Saving Blood Collection Device

(71) Applicant: Becton, Dickinson and Company, Franklin Lakes, NJ (US)

(72) Inventors: Benjamin P. Hopwood, West Valley City, UT (US); Weston F. Harding, Lehi, UT (US); BreAnna Eileen Lackey, West Valley City, UT (US)

(21) Appl. No.: 18/135,837

(22) Filed: Apr. 18, 2023

Related U.S. Application Data
(60) Provisional application No. 63/332,476, filed on Apr. 19, 2022.

(51) Int. Cl. A61B 5/15

(52) U.S. Cl. CPC A61B 5/15003; A61B 5/150221; A61B 5/150992

(57) ABSTRACT

A blood collection assembly couplable to a vascular access device, the assembly including a blood collection device having a first branch, a second branch, a third branch, and a channel configured for fluid communication therebetween, and a syringe having a syringe body, a plunger, and a stopper, wherein the plunger and the stopper are compressible and retractable within the syringe body, and wherein the syringe is coupled to the first branch of the blood collection device. The assembly also includes a testing sample collection device coupled to the second branch of the blood collection device, as well as an access portion coupled to the third branch of the blood collection device and configured to provide fluid communication between the vascular access device and the blood collection device.

### CROSS-REFERENCE TO RELATED APPLICATION

The present application claims priority to United States Provisional Application Serial No. 63/332,476, entitled “Blood Saving Blood Collection Device”, filed Apr. 19, 2022, the entire disclosure of which is hereby incorporated by reference in its entirety.

### BACKGROUND OF THE INVENTION

### Field of the Invention

The present disclosure generally relates to blood sampling with vascular access devices. More specifically, the present disclosure relates to blood sampling configurations allowing for a return of “waste blood” sample to the patient via a vascular access device such as, e.g., IV catheters, midlines, and central lines.

### Description of Related Art

Blood sampling is a common health care procedure involving the withdrawal of at least a drop of blood from a patient. Blood samples are commonly taken from hospitalized, homecare, and emergency room patients either by finger stick, heel stick, or venipuncture. Once collected, blood samples are analyzed via one or more blood test levels.

Despite the rapid advancement in point-of-care testing and diagnostics, blood sampling techniques have remained relatively unchanged. Blood samples are frequently drawn using hypodermic needles, or vacuum tubes coupled to a proximal end of a needle or a catheter assembly (i.e., a vascular access device). In some instances, clinicians collect blood from a catheter assembly using a needle and syringe that is inserted into the catheter to withdraw blood from a patient through the inserted catheter. Typically, blood draws using an existing vascular access device require a portion of “waste blood” to be disposed of after collection. Loss of such waste blood may be undesirable for a variety of reasons, including patient size (e.g., neonates), severe anemia, frequency of blood draw, previous blood loss, and/or other complicating factors.

Accordingly, in order to avoid wasted blood sample, some clinicians opt for sample collection via conventional venipuncture blood draws, which does not involve the collection of waste blood. However, because venipuncture blood draws require a needle stick each time a sample is to be collected, patient pain and anxiety may be a concern, particularly when numerous samples are needed over time.

### SUMMARY OF THE INVENTION

Accordingly, the present disclosure generally relates to blood collection devices used for blood draw via vascular access devices and related assemblies, systems, and methods. The blood collection devices are configured to draw a waste blood sample, temporarily store the waste blood sample, and return the waste blood back to the patient via the vascular access device.

In accordance with an embodiment of the present disclosure, a blood collection assembly couplable to a vascular access device is disclosed. The blood collection assembly may include a blood collection device having a first branch, a second branch, a third branch, and a channel configured for fluid communication between the first branch, the second branch, and the third branch, and a syringe having a syringe body, a plunger, and a stopper, wherein the plunger and the stopper are compressible and retractable within the syringe body, and wherein the syringe is coupled to the first branch of the blood collection device. The assembly may also include a testing sample collection device coupled to the second branch of the blood collection device, and an access portion coupled to the third branch of the blood collection device and configured to provide fluid communication between the vascular access device and the blood collection device.

In some embodiments, the blood collection device further includes a valve member, wherein rotation of the rotatable valve member switches fluid communication of the channel from communication between the first branch and the third branch to communication between the second branch and the third branch.

In some embodiments, the channel is a T-shaped channel.

In some embodiments, the valve member is selectively rotatable via a rotatable knob.

In some embodiments, the testing sample collection device includes a luer lock access device.

In some embodiments, the testing sample collection device includes a male-male luer connector.

In some embodiments, the access portion comprises flexible tubing and a luer access connector positioned on a distal end of the flexible tubing.

In some embodiments, the blood collection device further includes a compressible bulb positioned proximate the third branch and in fluid communication with the channel.

In some embodiments, the testing sample collection device includes a compressible bulb device, and the compressible bulb device is removably coupled to the second branch of the blood collection device.

In some embodiments, the compressible bulb device includes a nozzle and a connection interface, wherein the connection interface is configured to couple the compressible bulb device to an exterior surface of the second branch and the nozzle is configured to extend into the second branch so as to be in fluid communication with the channel.

In some embodiments, the blood collection device further includes a one-way valve positioned within the second branch, wherein the one-way valve is configured to close when the compressible bulb device is detached from the blood collection device and to open when the compressible bulb device is attached to the blood collection device.

In some embodiments, the nozzle of the compressible bulb device is configured to penetrate the one-way valve.

In some embodiments, the syringe includes an air trap portion.

According to another aspect of the present disclosure, a blood testing and diagnostic platform is disclosed, the blood testing and diagnostic platform including a point-of-care test device configured to be fluidly coupled to a vascular access device, a coupling member extending between the point-of-care test device and the vascular access device, and a waste blood reservoir disposed within the point-of-care test device, wherein waste blood reservoir is fluidly coupled to the coupling member via a first branch. The platform also may include a blood testing area disposed within the point-of-care test device, wherein the blood testing area is fluidly coupled to the coupling member via a second branch, and a valve provided in the coupling member, wherein the valve is switchable so as to selectively open and close the first branch and the second branch.

In some embodiments, the point-of-care test device further includes an integrated blood testing portion.

In some embodiments, the integrated blood testing portion includes at least one of a chemical test and an electronic sensor-based test.

In some embodiments, the blood testing area is removable from the point-of-care device.

In some embodiments, the point-of-care device is configured to both draw a waste blood sample into the waste blood reservoir before collection of a testing blood sample and infuse the waste blood sample back into a patient after a testing blood sample is collected in the blood testing area.

In accordance with another aspect of the present disclosure, a method of collecting a blood sample from a patient via a vascular access device is disclosed. The method may include providing a blood collection assembly, the blood collection assembly having a blood collection device including a first branch, a second branch, a third branch, and a channel configured for fluid communication between the first branch, the second branch, and the third branch, a syringe including a syringe body, a plunger, and a stopper, wherein the plunger and the stopper are compressible and retractable within the syringe body, and wherein the syringe is coupled to the first branch of the blood collection device, a testing sample collection device coupled to the second branch of the blood collection device, and an access portion coupled to the third branch of the blood collection device and configured to provide fluid communication between the vascular access device and the blood collection device. The method may further include retracting the plunger of the syringe to draw a waste blood sample into the syringe body, collecting a testing blood sample via the testing sample collection device, and compressing the plunger of the syringe to infuse the waste blood sample back into the patient.

In some embodiments, the method further includes switching a valve member within the blood collection device after drawing the waste blood sample into the syringe body and before collecting the testing blood sample.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a perspective view of a blood collection assembly in accordance with an aspect of the present disclosure;

FIG. 2 is a perspective view of a blood collection device in accordance with another aspect of the present disclosure;

FIG. 3 is a top plan view of the blood collection device of FIG. 2 in a first configuration;

FIG. 4 is a cross-sectional view of a blood collection assembly in accordance with another aspect of the present disclosure;

FIG. 5 is a cross-sectional view of a compressible bulb device usable with the blood collection assembly of FIG. 4;

FIG. 6 is a cross-sectional view of a syringe usable with the blood collection assembly of FIG. 4; and

FIG. 7 is a schematic view of a blood testing and diagnostic platform in accordance with another aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe in preparation for or during use. Similarly, in this application, the terms “in the distal direction” and “distally” mean in the direction toward a vascular access device, and the terms “in the proximal direction” and “proximally” mean in the direction away from the vascular access device.

While not shown or described herein, it is to be understood that the blood collection devices described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood draw devices for use with PIVCs. However, embodiments of the present disclosure equally extend to use with other catheter devices.

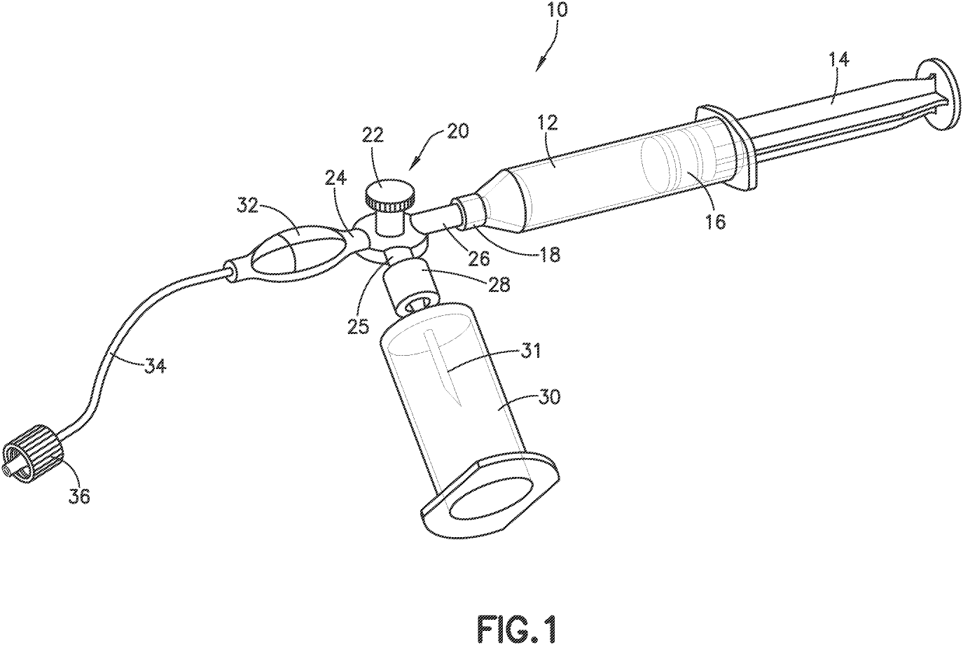

Referring to FIGS. 1-3, a blood collection assembly 10 in accordance with an aspect of the present disclosure is illustrated. Blood collection assembly 10 includes a blood collection device 20 configured to be coupled to a vascular access device such as, e.g., a PIVC. As will be described in further detail below, the blood collection device 20 enables a waste blood sample to be collected, followed by collection of a testing blood sample via, e.g., a luer lock access device (LLAD), a point-of-care testing device, etc., and then for the waste blood sample to be returned to the patient via the vascular access device.

As is shown in FIGS. 1-3, the blood collection device 20 may be configured as a T-shaped connector member having a first branch 24, a second branch 25, and a third branch 26. However, while blood collection device 20 is illustrated as T-shaped, it is to be understood that other configurations are possible (e.g., Y-shaped), and blood collection device 20 is not limited to three branches. Blood collection device 20 further includes a user-positionable valve member 22 disposed at an intersection of branches 24, 25, 26. The valve member 22 may be configured as a stopcock valve or any other appropriate switchable valve. In some embodiments, valve member 22 comprises a rotatable knob configured for at least 90° rotation, with rotation of the valve member 22 configured to rotate a T-shaped channel 23 formed within the valve member 22 between at least two positions, wherein a first position fluidly couples branches 24 and 26 (as is shown in FIG. 3) and a second position fluidly couples branches 24 and 25. While FIGS. 1-3 illustrate valve member 22 as a rotatable knob, it is to be understood that any appropriate valve configuration capable of selectively switching fluid access between branches 24, 25, 26 may be utilized in accordance with the present disclosure.

In some embodiments, the first branch 24 of blood collection device 20 may be coupled to a length of flexible tubing 34, with a connector 36 disposed at the distal end of tubing 34. The connector 36 may be any appropriate connector such as, e.g., a luer access connector capable of removably coupling the blood collection assembly 10 to a vascular access device (not shown) to enable fluid passage between the vascular access device and the blood collection device 20. In some embodiments, the tubing 34 may be tuned in length and diameter so as to prevent hemolysis. Alternatively, in other embodiments, the tubing 34 may be omitted, and the blood collection device 20 may be coupled substantially directly to the vascular access device.

Blood collection assembly 10 further includes a syringe 12 having a plunger 14 and stopper 16 slidably disposed therein for manual actuation by a clinician or other user. The syringe 12 includes a tip portion 18 configured to provide an inlet/outlet from a fluid chamber formed within the syringe 12. While not shown, the chamber of syringe 12 may contain a chemical stabilizer (e.g., heparin, citrate, etc.) to substantially prevent clotting of blood stored therein. The tip portion 18 is configured to be removably couplable to the third branch 26 via any appropriate connection method such as, e.g., a press-fit connection, a luer connection, etc. Additionally, while not shown, in some embodiments, the syringe 12 may include, e.g., one or more features to prevent the plunger 14 from being fully removed, features to enable turbulent mixing of blood and the chemical stabilizer(s), a mechanically-activated vacuum section to enable passive blood collection, and/or a low-pressure, two-way septum/valve configured to isolate the chamber/reservoir of the syringe.

Referring to FIG. 1, in some embodiments, the blood collection assembly 10 may also include a blood sample collection interface such as, e.g., a luer lock access device 30. In some embodiments, the luer lock access device 30 is removably couplable to the second branch 25 of the blood collection device 20 via a luer lock hub 28. However, in other embodiments, the luer lock access device 30 may be integrated with the blood collection device 20. As shown, the luer lock access device 30 is configured to receive a blood collection container such as, e.g., a BD ® VACUTAINER blood collection tube. The luer lock access device 30 includes a needle 31 in fluid communication with the luer lock hub 28 so as to allow fluids to selectively pass from the blood collection device 20 to a blood collection tube disposed within the luer lock access device 30. Although not shown, it is to be understood that the needle 31 may be substantially surrounded by a rubber sheath to protect from needle stick injuries. The luer lock access device 30 may include a holder sized and configured to accommodate e.g., a BD VACUTAINER^{®} blood collection tube. In response to the blood collection tube pushing the sheath distally towards the luer lock hub 28, the needle 31 may pierce the sheath, and the sharp proximal tip of the needle 31 may be inserted into the blood collection tube so as to receive a blood sample via the venous access device, with the patient’s venous pressure being sufficient to draw the sample into the evacuated blood collection tube.

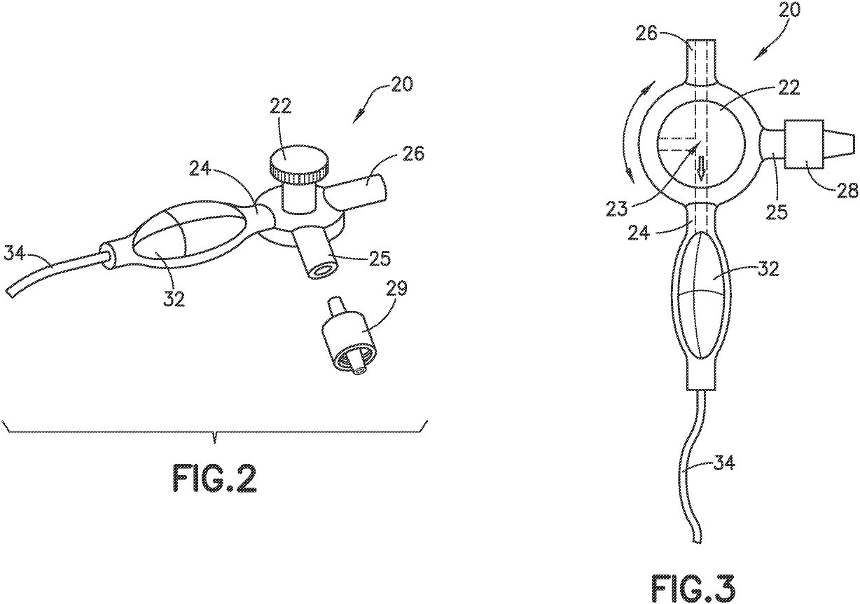

Alternatively, referring to FIG. 2, in some embodiments, the second branch 25 of blood collection device 20 may be coupled to other fittings such as, e.g., a male-male luer connector 29. In this way, the blood collection assembly 10 is not limited to blood collection via a luer lock access device and an associated blood collection tube, but may instead be coupled to other sample collection devices such as, e.g., small-sample dispensers for use in point-of-care testing, etc. In order to dispense the small-volume blood sample via the connector 29, the blood collection device 20 may include a compressible bulb 32. When the compressible bulb 32 is pressed by a clinician or other user, a small volume of blood may be dispensed from the second branch 25, with the small volume of blood being deliverable to, e.g., a point-of-care testing device coupled to (or otherwise positioned near) the connector 29. In this way, the blood collection device 20 may be usable for both large-volume sample collection (via a luer lock access device 30 and associated evacuated tube, as shown in FIG. 1) and small-volume sample collection. In some embodiments, the compressible bulb 32 may include a valve (not shown) to better enable dispensing.

Referring still to FIGS. 1-3, operation of the blood collection assembly 10 when coupled to a vascular access device will be described. First, with the valve member 22 positioned such that the first branch 24 and third branch 26 are fluidly coupled to one another via the channel 23 and the second branch 25 is isolated therefrom (i.e., the configuration shown in FIG. 3), the clinician or other user utilizes the syringe 12 to draw a waste blood sample by retracting the plunger 14 until a desired volume of waste blood is collected in the chamber of syringe 12. As noted above, the syringe 12 may contain a chemical stabilizer (e.g., heparin, citrate, etc.) such that the waste blood stored therein does not clot during the procedure. Additionally and/or alternatively, the syringe 12 and/or blood collection device 20 may include a mechanical or electrical timed lock (not shown) to prevent release/injection of any blood held within syringe 12 over a predetermined maximum time limit.

After a desired volume of waste blood is drawn into the syringe 12, the clinician may then rotate or otherwise actuate the valve member 22 such that the first branch 24 and second branch 25 of blood collection device 20 are in fluid communication via the channel 23, leaving the third channel 26 (and, thus, the waste blood held within syringe 12) isolated therefrom. The clinician can then initiate a blood draw and sample collection from the vascular access device into, e.g., a collection tube via the luer lock access device 30, a small-sample collection device for use in point-of-care testing, etc. As detailed above, in some embodiments, the clinician may compress the bulb 32 integrated into the blood collection device 20 in order to dispense a small-volume sample from the second branch 25.

Once the desired testing blood sample is collected via any appropriate means, the clinician may then rotate the valve member 22 to its initial position, with the first branch 24 and third branch 26 again being fluidly coupled to one another via the channel 23 and the second branch 25 being isolated therefrom. With valve member 22 returned to this position, the clinician may then depress the plunger 14 of syringe 12, which infuses the waste blood held within syringe 12 through the blood collection device 20 and back into the patient via the vascular access device. In this way, the waste blood held within syringe 12 does not need to be discarded, which may be desirable in a variety of circumstances where further loss of blood is to be avoided such as, e.g., small patient size (e.g., neonates, infants, etc.), patients with severe anemia, high frequency of blood draws, previous blood loss, and/or other complicating factors.

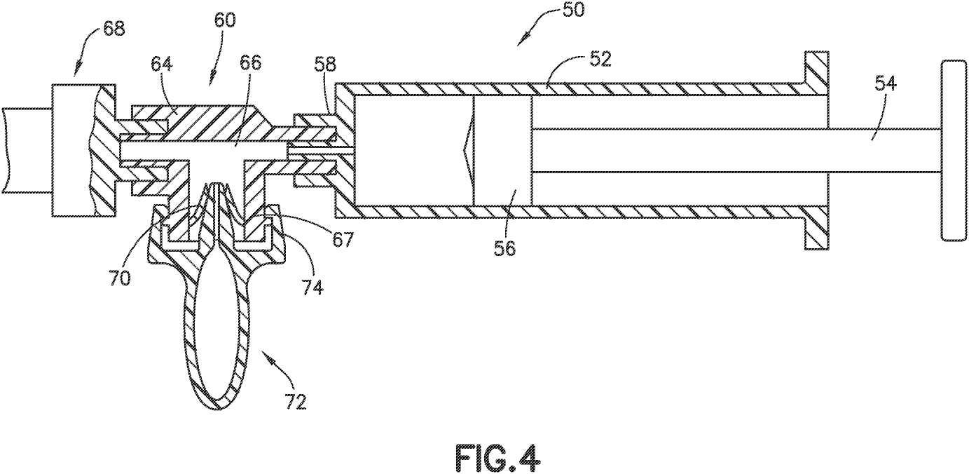

Next, referring to FIGS. 4-6, a blood collection assembly 50 in accordance with another aspect of the present disclosure is illustrated. Unlike blood collection assembly 10 described above with respect to FIGS. 1-3, which utilized a valve member for selective fluid coupling of a syringe to a venous access device, blood collection assembly 50 necessitates no user-actuatable valve for isolation of waste blood held in the syringe.

Blood collection assembly 50 includes a blood collection device 60 configured to be coupled to a vascular access device such as, e.g., a PIVC. The blood collection device 60 enables a waste blood sample to be collected, followed by collection of a testing blood sample via, e.g., a removable compressible bulb, and then for the waste blood sample to be returned to the patient via the vascular access device. The blood collection device 60 may be configured as a T-shaped connector member having a first branch 62, a second branch 64, and a third branch 67. However, while blood collection device 60 is illustrated as T-shaped, it is to be understood that other configurations are possible (e.g., Y-shaped), and blood collection device 60 is not necessarily limited to three branches. A channel 66 is formed within the blood collection device 60, with the channel 66 providing a fluid connection between each of the first branch 62, the second branch 64, and the third branch 67.

In some embodiments, the second branch 64 of blood collection device 60 may be coupled to a vascular access device (not shown) via a luer access device 68, which enables fluid passage between the vascular access device and the blood collection device 60. In some embodiments, tubing may extend between the luer access device 68 and the vascular access device, and the tubing may be tuned in length and diameter so as to prevent hemolysis. Alternatively, in other embodiments, the tubing may be omitted, and the blood collection device 60 may be coupled substantially directly to the vascular access device via the luer access device 68.

Blood collection assembly 50 further includes a syringe 52 having a plunger 54 and stopper 56 slidably disposed therein for manual actuation by a clinician. The syringe 52 includes a tip portion 58 configured to provide an inlet/outlet from a fluid chamber formed within the syringe 52. While not shown, the chamber of syringe 52 may contain a chemical stabilizer (e.g., heparin, citrate, etc.) to substantially prevent clotting of blood stored therein. The tip portion 58 is configured to be removably couplable to the first branch 62 of the blood collection device 60 via any appropriate connection method such as, e.g., a press-fit connection, a luer connection, etc. Additionally, while not shown, in some embodiments, the syringe 52 may include, e.g., one or more features to prevent the plunger 54 from being fully removed, features to enable turbulent mixing of blood and the chemical stabilizer(s), a mechanically-activated vacuum section to enable passive blood collection, and/or a low-pressure, two-way septum/valve configured to isolate the chamber/reservoir of the syringe.

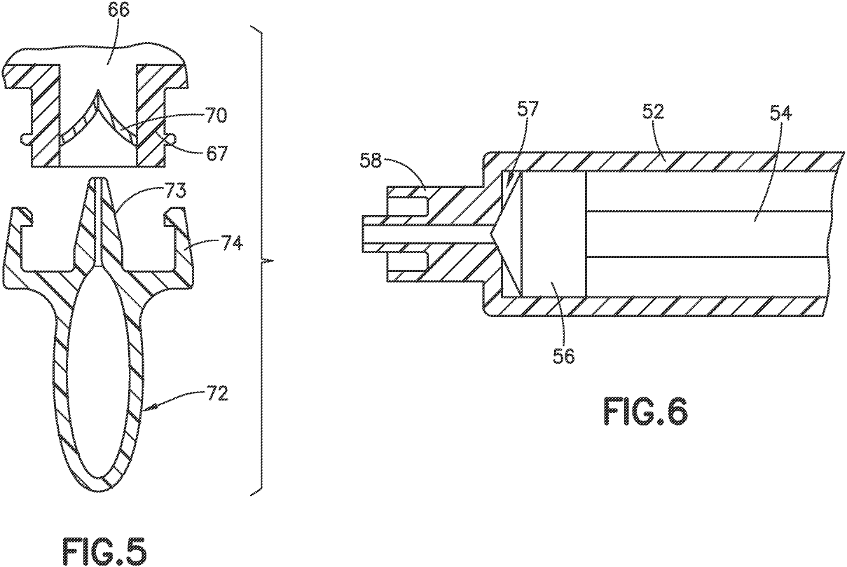

Referring still to FIGS. 4 and 5, blood collection assembly 50 further includes a compressible bulb device 72. The compressible bulb device 72 is configured to be removably couplable to the third branch 67 of the blood collection device 60 by way of a connection interface 74. The connection interface 74 may be any appropriate interface such as, e.g., a luer fitting, a snap-fit connection, a threaded connection, a press-fit connection, etc. The compressible bulb device 72 includes a nozzle portion 73 extending from a distal end thereof, wherein the nozzle portion 73 provides an inlet and outlet into the compressible bulb device 72.

As is shown in FIG. 5, the third branch 67 may include a one-way valve 70, with one-way valve 70 being normally closed when the compressible bulb device 72 is removed from connection with the third branch 67. In this way, fluid is able to pass between the first branch 62 and the second branch 64 of blood collection device 60 without leakage from third branch 67. However, when compressible bulb device 72 is coupled to the third branch 67 (as is shown in FIG. 4), the nozzle portion 73 of compressible bulb device 72 is configured to penetrate the one-way valve 70, thereby bringing the interior chamber of compressible bulb device 72 into fluid communication with the channel 66 of the blood collection device 60.

Operation of the blood collection assembly 50 in accordance with one aspect of the present disclosure will now be described. First, with the compressible bulb device 72 removed from the third branch 67 such that the one-way valve is closed (as is shown in FIG. 5), the clinician utilizes the syringe 52 to draw a waste blood sample by retracting the plunger 54 until a desired volume of waste blood is collected in the chamber of syringe 52. As noted above, the syringe 52 may contain a chemical stabilizer (e.g., heparin, citrate, etc.) such that the waste blood stored therein does not clot during the procedure. Additionally and/or alternatively, the syringe 52 and/or blood collection device 60 may include a mechanical or electrical timed lock to prevent release/injection of any blood held within syringe 52 over a predetermined maximum time limit.

After a desired volume of waste blood is drawn into the syringe 52, the clinician may then attach the compressible bulb device 72 to the third branch 67 such that the compressible bulb device 72 is in fluid communication with the channel 66. With the compressible bulb device 72 attached, the clinician may then squeeze or otherwise compress the compressible bulb device 72, allowing a vacuum to be applied to collect a small volume of blood from the venous access device within the compressible bulb device 72. Due to limited reflux from the chamber of the syringe 52, waste blood stored therein is not drawn into the compressible bulb device 72. The clinician may then detach the compressible bulb device 72 from the blood collection device 60, allowing the compressible bulb device 72 to immediately dispense small volume blood samples for point-of-care testing. Alternatively, in some embodiments, the compressible bulb device 72 may be transported and stored for future testing. The clinician may acquire any desired number of blood samples using one or more removable compressible bulb devices 72.

Once the desired testing blood sample(s) is/are collected via one or more compressible bulb devices 72, the clinician may then remove the compressible bulb device 72 such that the first branch 62 and second branch 64 are again fluidly coupled to one another via the channel 66, with the third branch 67 being closed by the one-way valve 70. The clinician may then depress the plunger 54 of syringe 52, which infuses the waste blood held within syringe 52 through the blood collection device 60 and back into the patient via the vascular access device. In this way, the waste blood held within syringe 52 does not need to be discarded, which may be desirable in a variety of circumstances where further loss of blood is to be avoided such as, e.g., small patient size (e.g., neonates, infants, etc.), patients with severe anemia, high frequency of blood draws, previous blood loss, and/or other complicating factors.

Still referring to FIGS. 4 and 5, and also referring to FIG. 6, operation of the blood collection assembly 50 in accordance with another aspect of the present disclosure will now be described. In some embodiments, the compressible bulb device 72 may be pre-attached to the blood collection device 60 and is present even when a waste blood sample is being collected. In such embodiments, the clinician again utilizes the syringe 52 to draw a waste blood sample by retracting the plunger 54 until a desired volume of waste blood is collected in the chamber of syringe 52. The retraction of plunger 54 also acts to draw air out of the attached compressible bulb device 72 and into the syringe 52.

Once a desired volume of waste blood is collected, the clinician stops retraction of the plunger 54, which allows the compressible bulb device 72 to automatically re-inflate and fill with blood via the venous access device. The clinician may then detach the compressible bulb device 72 from the blood collection device 60, allowing the compressible bulb device 72 to immediately dispense small volume blood samples for point-of-care testing, or be transported and stored for future testing. With the compressible bulb device 72 removed, the clinician may then depress the plunger 54 of syringe 52, which infuses the waste blood held within syringe 52 through the blood collection device 60 and back into the patient via the vascular access device.

As noted above, retraction of the plunger 54 while the compressible bulb device 72 is coupled to the blood collection device 60 results in air within the compressible bulb device 72 being drawn into the chamber of the syringe 52. In order to prevent that air from entering the patient’s bloodstream when the waste blood is returned to the patient via depression of the plunger 54, the syringe 52 (in conjunction with the shape of stopper 56) may be configured to have an air trap portion 57, as is shown in FIG. 6. Accordingly, when the plunger 54 is fully depressed to return the waste blood sample to the patient’s vasculature, the air trap portion 57 holds any air previously withdrawn from the compressible bulb device 72 in the syringe 52, thereby preventing that air from entering the patient’s bloodstream.

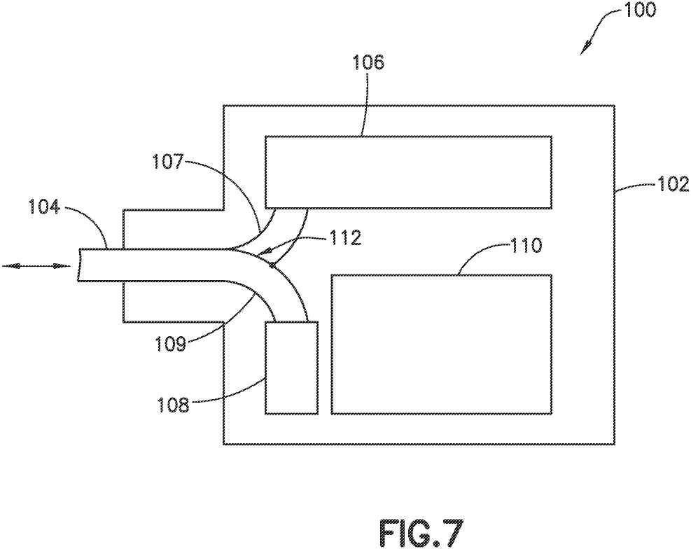

Referring now to FIG. 7, a blood testing and diagnostic platform 100 in accordance with another aspect of the present disclosure is illustrated. In some embodiments, blood testing and diagnostic platform 100 includes a point-of-care test device 102, with point-of-care test device 102 configured to be fluidly coupled to a vascular access device (not shown) via a coupling member 104. Coupling member 104 may be any appropriate coupler or combination of connectors and/or tubing.

The point-of-care test device 102 may include a waste blood reservoir 106, wherein waste blood reservoir 106 is fluidly coupled to the coupling member 104 via a first branch 107. Additionally, the point-of-care test device 102 may include a blood testing area 108. In some embodiments, the blood testing area 108 is integrated within the point-of-care test device 102. In other embodiments, the blood testing area 108 is removable from the point-of-care test device 102 and is therefore replaceable. The blood testing area 108 is fluidly coupled to the coupling member 104 via a second branch 109. A valve 112 is provided, with the valve 112 being selectively switchable so as to selectively open/close the first branch 107 and the second branch 109. In this way, fluid connection between the vascular access device and the waste blood reservoir 106 and the blood testing area 108 may alternate based on the position of the valve 112. The valve 112 may be actuated by any appropriate method, including manual switching, automatic (electrical) switching, etc.

Proximate and/or coupled to the testing area 108, the point-of-care test device 102 may include an integrated blood testing portion 110. The blood testing portion 110 may include any appropriate point-of-care testing feature such as, e.g., chemical test(s), electronic sensor-based test(s), and/or other diagnostic test(s).

Operation of the blood testing and diagnostic platform 100 in accordance with one aspect of the present disclosure will now be described. First, with the coupling member 104 connected to a vascular access device, the valve 112 is set such that the first branch 107 leading to the waste blood reservoir 106 is opened, while the second branch 109 leading to the blood testing area 108 is closed. Then, a desired volume of waste blood sample is drawn into the waste blood reservoir 106 via any appropriate method, e.g., a pump, a syringe, etc. The waste blood reservoir 106 may contain a chemical stabilizer (e.g., heparin, citrate, etc.) such that the waste blood stored therein does not clot during the procedure. Additionally and/or alternatively, the blood testing and diagnostic platform 100 may include a mechanical or electrical timed lock to prevent release/injection of any blood held within waste blood reservoir 106 over a predetermined maximum time limit.

After a desired volume of waste blood is drawn into the waste blood reservoir 106, the valve 112 is switched such that the second branch 109 leading to the blood testing area 108 is opened, while the first branch 107 is closed. A blood sample may then be collected in/on the blood testing area 108. In some embodiments, testing and/or diagnostics of the blood collected by blood testing area 108 is performed at the integrated blood testing portion 100. In other embodiments, the blood testing area 108 may be removed from the blood testing and diagnostic platform 100 after testing via the integrated blood testing portion 100 and/or to conduct testing remotely. If blood testing area 108 is removable, a removed blood testing area 108 may be replaced after blood collection and testing.

Once the desired testing blood sample(s) is/are collected in/on blood testing area 108 and testing by integrated blood testing portion 100 is completed, the valve 112 may again be switched such that the first branch 107 is opened, while the second branch 109 is closed. In this position, the waste blood held within the waste blood reservoir 106 can be infused back into the patient via the vascular access device. In this way, the waste blood held within the waste blood reservoir 106 does not need to be discarded, which may be desirable in a variety of circumstances where further loss of blood is to be avoided.

While several embodiments of blood collection assemblies configured for the collection (and return) of waste blood were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A blood collection assembly couplable to a vascular access device, the blood collection assembly comprising:
a blood collection device comprising a first branch, a second branch, a third branch, and a channel configured for fluid communication between the first branch, the second branch, and the third branch;
a syringe comprising a syringe body, a plunger, and a stopper, wherein the plunger and the stopper are compressible and retractable within the syringe body, and wherein the syringe is coupled to the first branch of the blood collection device;
a testing sample collection device coupled to the second branch of the blood collection device; and
an access portion coupled to the third branch of the blood collection device and configured to provide fluid communication between the vascular access device and the blood collection device.

2. The blood collection assembly of claim 1, wherein the blood collection device further comprises a valve member, wherein rotation of the rotatable valve member switches fluid communication of the channel from communication between the first branch and the third branch to communication between the second branch and the third branch.

3. The blood collection assembly of claim 2, wherein the channel is a T-shaped channel.

4. The blood collection assembly of claim 2, wherein the valve member is selectively rotatable via a rotatable knob.

5. The blood collection assembly of claim 1, wherein the testing sample collection device comprises a luer lock access device.

6. The blood collection assembly of claim 1, wherein the testing sample collection device comprises a male-male luer connector.

7. The blood collection assembly of claim 1, wherein the access portion comprises flexible tubing and a luer access connector positioned on a distal end of the flexible tubing.

8. The blood collection assembly of claim 1, wherein the blood collection device further comprises a compressible bulb positioned proximate the third branch and in fluid communication with the channel.

9. The blood collection assembly of claim 1, wherein the testing sample collection device comprises a compressible bulb device, and wherein the compressible bulb device is removably coupled to the second branch of the blood collection device.

10. The blood collection assembly of claim 9, wherein the compressible bulb device comprises a nozzle and a connection interface, wherein the connection interface is configured to couple the compressible bulb device to an exterior surface of the second branch and the nozzle is configured to extend into the second branch so as to be in fluid communication with the channel.

11. The blood collection assembly of claim 10, wherein the blood collection device further comprises a one-way valve positioned within the second branch, wherein the one-way valve is configured to close when the compressible bulb device is detached from the blood collection device and to open when the compressible bulb device is attached to the blood collection device.

12. The blood collection assembly of claim 11, wherein the nozzle of the compressible bulb device is configured to penetrate the one-way valve.

13. The blood collection assembly of claim 1, wherein the syringe comprises an air trap portion.

14. A blood testing and diagnostic platform comprising:
a point-of-care test device configured to be fluidly coupled to a vascular access device;
a coupling member extending between the point-of-care test device and the vascular access device;
a waste blood reservoir disposed within the point-of-care test device, wherein waste blood reservoir is fluidly coupled to the coupling member via a first branch;
a blood testing area disposed within the point-of-care test device, wherein the blood testing area is fluidly coupled to the coupling member via a second branch; and
a valve provided in the coupling member, wherein the valve is switchable so as to selectively open and close the first branch and the second branch.

15. The blood testing and diagnostic platform of claim 14, wherein the point-of-care test device further comprises an integrated blood testing portion.

16. The blood testing and diagnostic platform of claim 15, wherein the integrated blood testing portion comprises at least one of a chemical test and an electronic sensor-based test.

17. The blood testing and diagnostic platform of claim 14, wherein the blood testing area is removable from the point-of-care device.

18. The blood testing and diagnostic platform of claim 14, wherein the point-of-care device is configured to both draw a waste blood sample into the waste blood reservoir before collection of a testing blood sample and infuse the waste blood sample back into a patient after a testing blood sample is collected in the blood testing area.

19. A method of collecting a blood sample from a patient via a vascular access device, the method comprising:
providing a blood collection assembly, the blood collection assembly comprising:
a blood collection device comprising a first branch, a second branch, a third branch, and a channel configured for fluid communication between the first branch, the second branch, and the third branch,
a syringe comprising a syringe body, a plunger, and a stopper, wherein the plunger and the stopper are compressible and retractable within the syringe body, and wherein the syringe is coupled to the first branch of the blood collection device,
a testing sample collection device coupled to the second branch of the blood collection device, and
an access portion coupled to the third branch of the blood collection device and configured to provide fluid communication between the vascular access device and the blood collection device;
retracting the plunger of the syringe to draw a waste blood sample into the syringe body;
collecting a testing blood sample via the testing sample collection device; and
compressing the plunger of the syringe to infuse the waste blood sample back into the patient.

20. The method of claim 19, further comprising switching a valve member within the blood collection device after drawing the waste blood sample into the syringe body and before collecting the testing blood sample.
